(19) United States
(12) Patent Application Publication
(10) Pub. No.: US 2024/0366281 A1
(43) Pub. Date: Nov. 7, 2024

(54) PLASMA INSTRUMENT

(71) Applicant: Erbe Elektromedizin GmbH, Tuebingen (DE)

(72) Inventors: Martin WALZ, Rottenburg (DE); Sandra MOSER, Albstadt-Laufen (DE); Johannes HEYM, Tuebingen (DE); Fabian BEUTLER, Neckartenzlingen (DE)

(21) Appl. No.: 18/642,079

(22) Filed: Apr. 22, 2024

(30) Foreign Application Priority Data
May 4, 2023 (EP) 23171481.7

(51) Int. Cl. A61B 18/04; A61M 25/00

(52) U.S. Cl. CPC A61B 18/042; A61M 25/007

(57) ABSTRACT

An instrument according to the invention for plasma treatment of biological tissue comprises plasma exit windows in form of openings, which widen or taper in longitudinal direction. In doing so, inclined positions of webs result, which are present between the individual openings and which seamlessly monolithically connect a proximal section of the instrument head with a distal section. Due to the inclined position of the webs, the shading effect is minimized or eliminated.

This application claims priority to European Patent Application No. 23171481.7, filed May 4, 2023, the entirety of which is incorporated by reference herein.

The invention refers to a plasma instrument for influencing biological tissue, particularly for surgical treatment of human or animal tissue. The plasma instrument is particularly configured as laterally or radially operative instrument.

A plasma probe having a hose or a tube-like base body is known from EP 1 682 023 B1, through which a lumen extends from the proximal end to a distal end. The lumen is connected to a gas source. Through the lumen an electrical conductor extends, which is proximally connected to an electrical source. It projects distally out of the lumen. At its distal end the conductor serving as electrode, supports an insulating body, which is for example ball-shaped or disc-shaped, which defines a ring gap with the edge of the mouth of the lumen. Through this ring gap a plasma stream can exit in any arbitrary radial direction, depending on the direction in which the closest biological tissue is located, which is connected to the counter-pole of the electrical source.

Any force acting on the insulating body supported by the electrode has to be supported by the electrode and has to be transferred to the distal end of the hose.

Also, probes are known in which the distal end of the hose is provided with an end piece of ceramic having one or more lateral openings out of which a plasma jet may exit respectively. However, it thereby shows that the plasma jet tends to jump between the windows depending on where the closest biological electrically conductive tissue is located.

Additional plasma instruments for influencing biological tissue are known from the prior art. For example, DE 198 20 240 A1 as well as EP 1 297 082 A1 respectively disclose instruments having a hose-like base body through which a lumen extends from its proximal end to its distal end on which a head with one or multiple lateral windows is arranged. An electrical conductor extends through the lumen, which is connected to a central electrode at its distal end. In operation an electrode connected to an HF-voltage source ionizes the flowing gas, which then exits laterally as plasma stream. In an embodiment two slit-like plasma exit windows are provided that are axially arranged at different locations and slightly overlap with one another.

EP 3 831 291 A1 discloses an instrument for production of a plasma for influencing biological tissue. Again, the instrument comprises a hose-like body, which, at least in one embodiment, supports a head having a lateral opening on its distal end through which a plasma stream may exit.

The instrument known from U.S. Pat. No. 9,510,889 B2 comprises an axial opening at its distal end out of which an electrode projects. The electrode supports an insulating body at its distal end, which limits a ring-shaped radially opening slit with the remaining instrument. The plasma stream has unblocked radial exit around 360° at each circumferential location, wherein however the insulating body has to be supported by the electrode and has to be maintained in a stable manner.

Additional prior art can be taken from US 2021/0259756 A1, EP 3 422 981 A2, EP 3 372 183 A1, JP 2002-301088 A and GB 2 573 128 A.

It is the object of the invention to provide a robust plasma probe with uniform radial efficacy.

This object is solved by a probe according to claim 1:

The plasma instrument according to the invention is particularly configured as probe for the endoscopic application. However, as required it can also be configured as laparoscopic instrument or as instrument for the open surgical use.

The instrument comprises a longitudinal body configured as hose or tube through which longitudinally at least one lumen extends. The body can comprise one or more lumen, i.e. it can be a single or multiple lumen hose. It can also be configured as a single or multiple lumen tube.

The proximal end of the body or its lumen is connectable to a gas source, for example a respective apparatus, via which the instrument can be supplied with a gas, particularly an inert gas, such as argon. The apparatus or other gas source is configured to provide the gas for the instrument with the required pressure and in the required amount.

On the distal end of the body a head is provided, which comprises at least two openings connected with the one or more lumen, wherein the openings are separated from one another by a web. The web is orientated obliquely, i.e. not parallel, to the longitudinal direction of the instrument. Due to the inclined position of the web, each opening can comprise edges of different lengths at its distal or at its proximal end. For each web applies that its distal end is offset in circumferential direction compared to its proximal end. The circumferential direction is thereby defined by a circle around the longitudinal center axis of the head (and hose).

Preferably the openings are trapezoid- or triangular-shaped. Thereby further preferably, the sum of the long edges of all windows is longer than it would have been in the case of webs orientated longitudinally. Radial directions exist that are common to both openings. In doing so, a radial exiting plasma jet can be less jumpy and thus can transition by a smoother change from one window to another. The behavior of the instrument according to the invention with regard to the influence at the point of impact of the plasma jet is better to predict and easier to control for the user.

In a preferred embodiment the sum of the long edges of the windows is longer than the circumference of the head. In a preferred embodiment this applies, if the circumference is measured at the distal end of the windows in case of a head tapering in distal direction. Thereby, a large overlap of the openings is achieved, whereby a particularly smooth plasma transition is achieved.

According to the concept of the invention, the web is inclined in circumferential direction. Thereby multiple webs can be inclined in opposite orientation respectively, so that openings are formed, which alternatingly taper in distal direction and widen in distal direction, wherein the openings are arranged in series along the circumference of the head. Thereby all openings can end proximally at a uniform axial position. Also, the distal limitations of all windows can be located at the same axial position respectively. The centers of the areas of the individual openings can then be located on a zigzag line.

Preferably the inclined position of the web in circumferential direction has such an extent that the openings slightly overlap one another in axial and in circumferential direction. The transition of the plasma jet or spark from one opening to another is then particularly continual.

The openings can be configured triangular-shaped, triangular-shaped with rounded corners, trapezoid-shaped, preferably with rounded corners or also diamond-shaped. In all these cases the blocking of the plasma jet or spark by the webs is largely minimized.

Basically, the plasma jet can be created in different manners. For example, an electrode, which is connected to an electrical source by an electrical line can be arranged in the lumen in the proximity of the opening. The electrode can be arranged concentrically on the head and thus be positioned on its longitudinal center axis. The electrical line can extend through the lumen or can be inserted in the material of the body (hose). The electrical line is configured to be connected with one pole of an electrical generator at its proximal end in order to supply the electrode with current and voltage. Preferably, the generator is a high frequency generator. If only one electrode is present, the instrument is a monopolar instrument in which the counter electrode of the voltage source has to be connected to the patient. The current flow is then from the electrode through the plasma exiting from the opening to the tissue.

Alternatively, the plasma can also be created in another manner. For example, for this purpose at least one electrically insulated electrode, which can be incorporated in the head, for example, can be provided for generating a non-thermal plasma. Another electrode can also be incorporated in the head or can be arranged in the lumen. Such electrodes can be connected with the poles of an electrical high frequency generator via lines in order to create a so-called barrier discharge in the head.

Further details of advantageous embodiments of the invention defined by the claims and the description as well as the drawing. The drawings show:

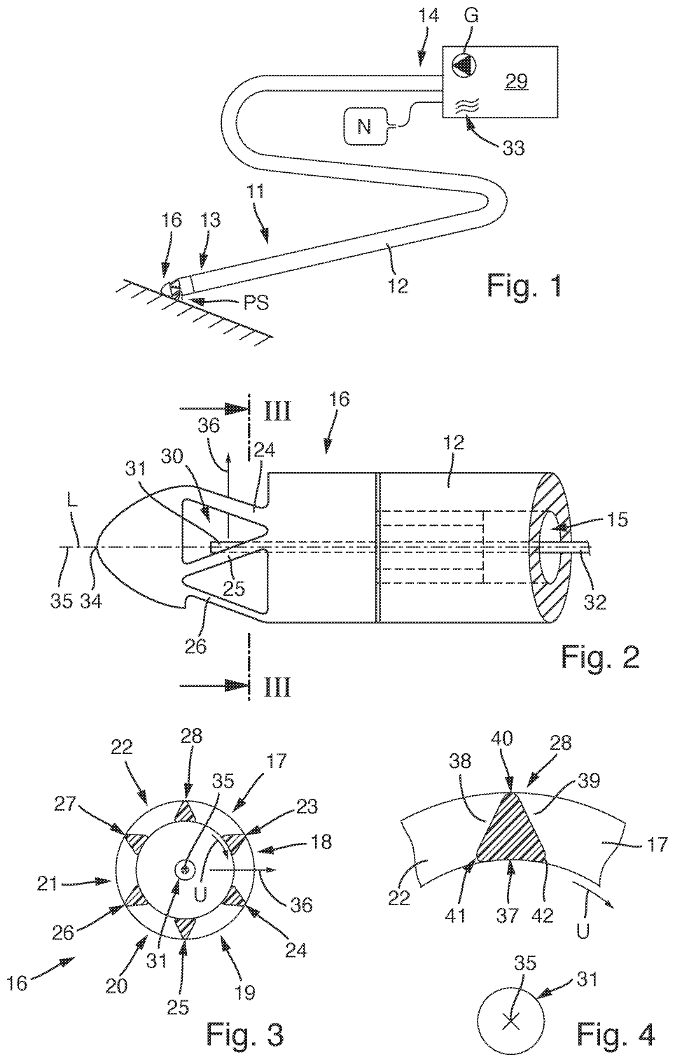

FIG. 1 shows an embodiment of a plasma instrument according to the invention connected to a supplying apparatus in a perspective basic illustration,

FIG. 2 shows the distal end of the plasma instrument according to FIG. 1 in enlarged side view, which is not intended to show scale,

FIG. 3 shows the plasma instrument according to FIGS. 1 and 2 cut along line III-III of FIG. 2,

FIG. 4 shows the plasma instrument according to FIGS. 2 and 3 cut along the cutting line III-III in part and highly enlarged,

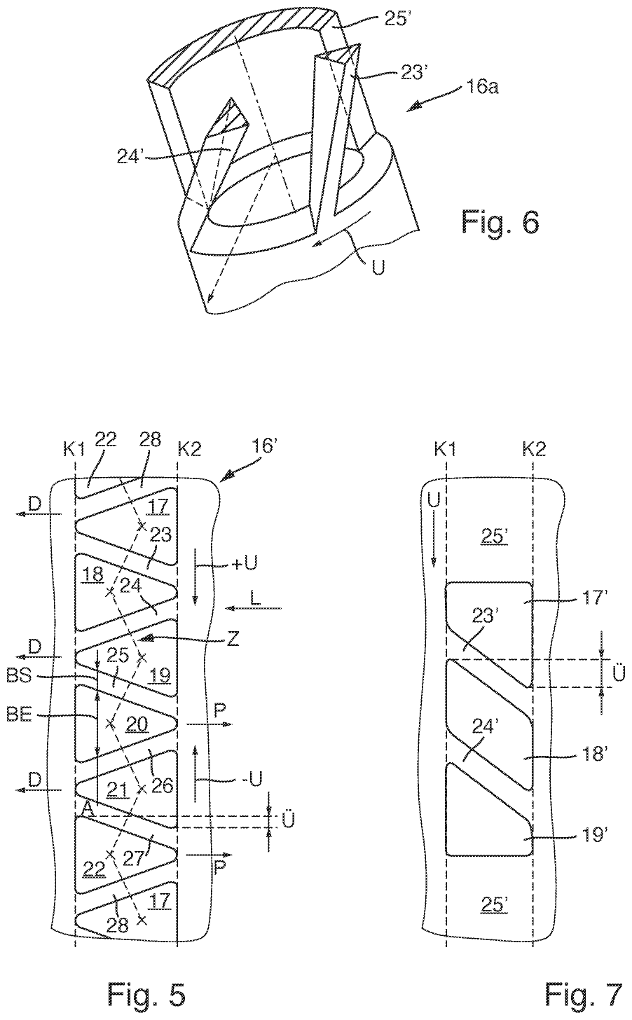

FIG. 5 shows the unwrapped depiction of the head apparent from FIG. 2 for illustration of the geometric relations,

FIG. 6 shows a modified embodiment of a plasma instrument according to the invention in a cut perspective illustration in part,

FIG. 7 shows an unwrapped depiction of the head according to FIG. 6 for illustration of the geometric relations,

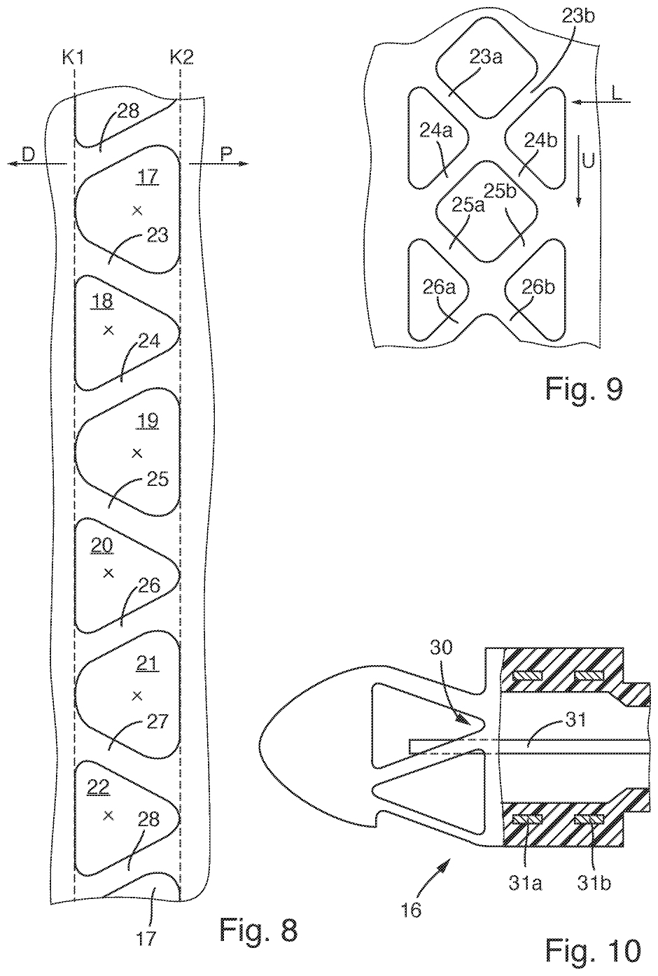

FIGS. 8 and 9 show unwrapped depictions of additional alternative embodiments of probes according to the invention and

FIG. 10 shows a plasma probe having at least two electrodes in an illustration of its head in part and partly cut.

FIG. 1 illustrates a plasma instrument 11 configured as flexible probe having a flexible hose as body 12. The body 12 comprises a distal end 13 and a proximal end 14 as well as a lumen 15 extending from the proximal end 14 to the distal end 13. The body 12 can comprise one single lumen 15, as illustrated in FIG. 2, or also multiple lumen. The body 12 is configured in a bendable or flexible manner, for example in order to be moved as probe through the working channel of an endoscope to the operation site of a patient. If the endoscope comprises a controllable end, the flexibility of the instrument allows to carry out a respective bending movement.

The instrument 11 can not only be provided as endoscopic usable probe, but also in another form, for example as laparoscopic instrument, whereby the body 12 is a rigid tube. Also, the instrument 11 can be configured for the open surgical use having a short body 12 and a hand piece provided at the proximal end.

On the distal end 13 of body 12 a head 16 is arranged, which comprises at least two, preferably however, multiple openings, for example six openings 17 to 22, as apparent from FIG. 3. Alternatively, also three, four, five, seven, eight or more openings can be provided. The openings 17 to 22 are separated by webs 22 to 28 from each other, which consist of heat resistant material, for example ceramic, such as preferably the entire head 16.

At the proximal end 14 lumen 15 is connected to an apparatus 29, which serves for supply of instrument 11. For example, the apparatus 29 can be configured as gas source G or can comprise such a gas source. For this purpose, it can be connected to a gas storage, for example a gas bottle or the like, and can comprise a controller, particularly for releasing and blocking, as necessary for dosage of the gas flow, with which the lumen 15 is supplied. The gas storage can contain particularly argon or also another inert gas, particularly an inert gas suitable for plasma creation. As necessary, apparatus 29 can also be configured for supply of active gases, i.e. reactive gases, aerosols, vapors or the like.

The lumen 15 is connected with the openings 17 to 22, so that gas flowing through the lumen to the head 16 can exit equally from all openings 17 to 22.

The instrument 11 comprises in addition at least one plasma creation device 30, for example in the form of an electrode 31, the distal end of which is positioned in the region of the openings 17 to 22. The electrode can be formed by a substantially cylindrical body of metal, the distal end of which is arranged in the region of the openings 17 to 22, preferably substantially in the center thereof. As illustrated, electrode 31 can have a circular cross-section or also a polygonal cross-section. Instead of a single electrode, also a bundle of electrical conductors can be provided, which contact one another or which are arranged in lateral distance to one another. Preferably, however not necessarily, electrode 31 has a diameter that is at least as large as a width of a web measured in the cross-section according to FIGS. 3 and 4 in circumferential direction U. In case of an electrode bundle, the diameter D is the outer diameter of the cross-section of the bundle.

An electrical conductor 32 can extend from the electrode 31 up to the proximal end 14 of instrument 11 and can be connected to the apparatus 29 there. The apparatus 29 can comprise an electrical source, for example in form of a high voltage high frequency generator 33, via which the electrode 31 can be supplied with an electrical voltage. The high frequency voltage has such an amount that it is sufficient for ionization of the gas stream supplied via lumen 15 in order to form a plasma. The voltage usually has an amount of multiple 100 Volts at a frequency above 100 KHz, preferably multiple 100 kHz, further preferably however below 5 MHz.

The special feature of the invention is the configuration of head 16, which has at least preferably a cross-section tapering toward the distal tip 34. The openings 17 to 22 and thus also the webs 23 to 22 can be entirely or partly arranged in the tapering section of head 16, which starts in FIG. 2 approximately at cutting line III-III. In the tapering section the diameter of head 16 decreases continuously up to the distal tip 34 where the head 16 ends in a curvature, preferably bluntly.

The individual openings 17 to 22 are preferably separated by webs 23 to 28, which are arranged inclined relative to a longitudinal direction L at least in circumferential direction. The longitudinal direction L is indicated in FIG. 2 by a longitudinal center axis 35, which forms a symmetry axis for the rotational symmetry of the head 16. The rotational symmetry is three-fold in the embodiment according to FIGS. 2 and 3, i.e. in case of a rotation of the head about 120° around the longitudinal center axis 35, the rotated head 16 is congruent with the non-rotated head 16.

The openings 17 to 22 limited between the webs 23 to 28 are orientated radially relative to the longitudinal center axis 35. In FIG. 2 the opening direction of the opening 17 is marked by a dashed arrow 36. The openings 17 to 22 thus open transverse to the longitudinal center axis 35 and thus also transverse to the longitudinal direction L in radial direction or, as indicated by arrow 36, also slightly inclined relative to the radial direction. For determination of the opening direction, head 16 can be first considered without window as rotational body tapering toward the distal tip 34. As illustrated by FIG. 2, the tapering can increase toward the distal tip 34 so that the longitudinal section through the head 16 approximates a parabolic shape or a half ellipse. If on such a virtual head 16 without opening, an opening is drawn at the position provided for the respective opening 17 to 22 and if in the center of the area thereof the normal vector is indicated, the latter characterizes the direction of arrow 36.

The webs 23 to 28 preferably comprise a cross-section tapering in positive radial direction +R (radially outwardly), for example a triangular cross-section, which is illustrated in FIG. 4 by way of example of web 28. The latter is cut in the same cutting plane III-III, as indicated in FIG. 2 and illustrated in FIG. 3.

The cross-section of web 28 illustrated in FIG. 4 comprises a lateral surface 37 facing the longitudinal center axis 35 as well as two additional lateral surfaces 38, 39, laterally limiting the windows 22 and 17. The cross-section of the webs 23 to 28 are preferably substantially constant along each web. The lateral surfaces 38, 39 configured in a planar or also convexly rounded manner limit an acute angle with one another, which opens toward the longitudinal center axis. Thereby web 28 faces electrode 31 with its interior lateral surface 37. With its preferably rounded corner 40, web 28 points radially outwardly with view from the longitudinal center axis 35, i.e. away from the longitudinal center axis 35. The remaining corners 41, 42 are preferably rounded as well. The lateral surfaces 38, 39 can be planar or preferably slightly convexly rounded. The lateral surface 37 can be planar or as illustrated in FIG. 4 concave, as necessary however also convexly rounded.

FIG. 5 illustrates an unwrapped depiction 16′ of head 16, the ends of which overlap. As apparent the six windows 17 to 22 are triangular. Adjacent webs 23 to 28 are inclined in circumferential direction U oppositely in pairs respectively. Thereby the absolute values of the inclination angles, which the individual webs 23 to 28 limit with the circumferential direction U are equal. In other words, the inclination angle of each web 23 to 28 changes its sign along the circumferential direction from web to web. In doing so, the openings 17 to 22 obtain a triangular basic shape with more or less strongly rounded corners, wherein the bases of the triangles marked by openings 17 to 22 are positioned on circles K1, K2 that are parallel to one another having equal or slightly different diameters. The center points of the circles K1 and K2 are positioned on the longitudinal center axis 35. The tips of the triangles marked by openings 17 to 22 alternatingly point in distal direction D or proximal direction P. The substantially triangular openings 17 to 22 have centers of area, which can be positioned on a zigzag line Z, as indicated in FIG. 5.

The inclinations or inclination angles of webs 23 to 28 are dimensioned so that the openings 17 to 22 overlap in circumferential direction U. The circumferential direction is characterized by the progress of the circles K1, K2 illustrated here as straight lines, due to the unwrapped depiction. In FIG. 5 the overlap is illustrated by way of example of web 27 and the openings 21, 22. The corner of opening 21 positioned in front in positive circumferential direction +U and the opening of corner 22 located at the rear in positive circumferential direction +U overlap one another about the overlap absolute value U. The latter is at least zero and preferably larger than zero. In other words, if opening 21 is virtually moved in distal direction D and/or opening 22 is virtually moved in proximal direction P, the openings 21, 22 touch or overlap one another.

Each web comprises a width BS to be measured in circumferential direction U and each opening comprises a width BE to be measured in circumferential direction. FIG. 5 illustrates this by web 25 and opening 20 located adjacent to the wider end of opening 20 and thus circle K1. For the opening 19 this applies accordingly with circle K2. This means that the width BS of web 23 is preferably smaller than the width BE, at least of one adjacent opening. This applies at least at one axial position, wherein the width BS and the width BE are measured at the same axial position A. The mentioned condition for the widths BS and BE (BE>BS) applies respectively at least there where the openings have their largest extension in circumferential direction U.

In the embodiment according to FIGS. 1 to 5, webs 23 to 28 are in pairs inclined in and against the circumferential direction (i.e. in +U and −U). It is however also possible to incline webs 23′,24′ in the same orientation as illustrated based on an embodiment according to FIGS. 6 and 7. While instrument 11 described above is an instrument with 360°, all-round effectiveness instrument 11′ according to FIG. 6 is provided for tissue treatment only in a limited circumferential area. The head 16a comprises a wider section 25′, which is configured with or without inclination in circumferential direction and can be considered as curved wall, but however not as web. Three openings 17′,18′,19′ are obtained, which can be configured trapezoid-shaped or diamond-shaped. As apparent, trapezoid-shaped opening 17′ and diamond-shaped opening 18′ overlap in circumferential direction U again. The same applies for the opening 18′ and the trapezoid-shaped opening 19′. Apart therefrom, the above description applies on the basis of the already introduced reference signs.

Further modifications are possible. FIG. 8 therefore shows an unwrapped depiction of a modified head 16 for which the description of the embodiment according to FIGS. 1 to 5 applies fully accordingly apart from the special features indicated in the following:

The openings 17 to 22 are not configured in triangular-, but trapezoid-shaped manner. For this purpose, instead of the respective tip of each surface 17 to 22 pointing in distal direction D or proximal direction P, a strong rounding or also a short edge is provided, which transitions into the respective web 23 to 28 by two curvatures.

The embodiments described so far use openings 17 to 22 arranged between the two virtual circles K1, K2 in one single line. However, also arrangements in multiple lines are possible, as illustrated in FIG. 9. The special feature of this arrangement consisting in total of three lines of rounded triangular or quadrangular openings are again inclined webs 23a, 23b, 24a, 24b, 25a, 25b etc., which are inclined in or against circumferential direction U and are thus arranged non-parallel to the longitudinal direction L. Again, the individual openings overlap in circumferential direction with one another.

Another modification is possible with regard to the configuration of the plasma creation device 30. While in the embodiments described so far only one blank electrode 31 is provided as plasma creation device 30 in order to create a largely thermal plasma, as an alternative also one or multiple electrodes 31a, 31b arranged in electrically insulated manner can be provided for forming a barrier discharge. For example, the electrode 31a and/or 31b can be embedded as bowl- or ring-shaped electrodes in the material of head 16 or can be arranged in another insulated manner therein. The two poles of the generator can be connected with the insulated electrodes 31a, 31b in order to create a barrier discharge. In this case the electrode 31 can be omitted. Alternatively, one or both electrodes 31a, 31b can be connected to one pole of the generator and the electrode 31 that is uncovered or provided with an insulating layer can be connected with the other pole of the generator in order to create a barrier discharge. Such arrangements particularly serve for creation of non-thermal warm or cold plasma. The different plasma creation devices 30 described briefly herein can be combined with any of the above-described heads according to FIGS. 1 to 9.

The instrument 11 described so far operates as follows:

In operation instrument 11 is supplied with gas, for example argon, from the apparatus 29 so that it flows through lumen 15 and out of openings 17 to 22. In addition, the electrical source 33 is activated so that an electrical discharge occurs for ionization of the gas stream and for creation of plasma at the plasma creation device 30. Particularly in the monopolar variant in which one pole of the electrical source 33 is connected with the preferably uncovered electrode 31 and the other pole of the source 33 is connected with the patient, an electrical discharge forms between the electrode 31 and the biological tissue closest to the head 16, so that a plasma stream PS is established through the opening positioned inbetween, for example opening 19. This condition is illustrated in FIG. 1.

If now instrument 11 is moved, it can happen that the discharge, i.e. the plasma stream, finds its way rather through the adjacent window 18 or 20. The transition of the plasma stream from window 19 to window 18 or 20 is thereby continuous and smooth, because the windows overlap in circumferential direction with one another, particularly with viewing direction in longitudinal direction L, as obvious by way of example of FIG. 5. The obliquely orientated webs 23 to 28 do therefore not block the discharge, which remarkably reduces the volatility of the plasma jet, recognized as spark by the treating person, compared with instruments having webs orientated in longitudinal direction.

If a device with barrier discharge is used as plasma creation device 30 as illustrated according to FIG. 10, it can be achieved that the plasma is created in a ring-shaped area covering all of the openings 17 to 22 without gap, whereby shavings due to webs 23 to 28 are again minimized.

The instrument 11 according to the invention for plasma treatment of biological tissue comprises plasma exit windows in form of openings 17 to 22, which widen or taper in longitudinal direction. Each opening 17 to 22 comprises at least one edge extending obliquely relative to the longitudinal direction L, which can be formed by a flank 38, 39 of a web. Inclined positions of webs 23 to 28 present between the individual openings result, which seamlessly monolithically connect a proximal section of the instrument head 16 with a distal section. Due to the inclined position of the webs, its shaving effect is minimized or eliminated.

1. A plasma instrument for influencing biological tissue particularly for surgical treatment of human or animal patients, comprising:
a longitudinal body configured as a hose or a tube having a distal end and a proximal end as well as a lumen defining a longitudinal direction (L), which extends from the proximal end to the distal end and is configured to connect to a gas source (G), and
a head connected to the distal end comprising at least two openings connected to the lumen, which are separated from one another by a web orientated obliquely to the longitudinal direction (L).

2. The plasma instrument according to claim 1, further comprising a plasma creation device is arranged in the lumen or in the head.

3. The plasma instrument according to claim 2, wherein the plasma creation device comprises at least one electrode configured to connect to an electrical source.

4. The plasma instrument according to claim 3, wherein the electrode is a metallic non-insulated electrode configured to connect to one pole of the electrical source.

5. The plasma instrument according to claim 1, wherein the openings are orientated transverse to the longitudinal direction (L).

6. The plasma instrument according to claim 1 wherein the openings are orientated radially relative to the longitudinal direction (L).

7. The plasma instrument according to claim 1, wherein the web is arranged in a distally tapering section of the head.

8. The plasma instrument according to claim 1, wherein the web is inclined in circumferential direction (U) of the head.

9. The plasma instrument according to claim 8, wherein the web is further inclined distally toward a longitudinal center axis extending centered in longitudinal direction (L) in the head.

10. The plasma instrument according to claim 8, wherein the web is orientated obliquely relative to the longitudinal direction (L), so that the openings with view in longitudinal direction (L) adjoin one another or overlap one another.

11. The plasma instrument according to claim 1, wherein the head comprises multiple webs, which are arranged between the openings.

12. The plasma instrument according to claim 11, wherein adjacent webs are arranged inclined in pairs in opposite directions (+U, −U) relative to one another.

13. The plasma instrument according to claim 11, wherein the openings are configured in triangular-shaped manner with rounded corners.

14. The plasma instrument according to claim 1, wherein the openings are configured in trapezoid-shaped manner with rounded corners.

15. The plasma instrument according to claim 1, wherein the openings form a line extending around the entire circumference of the head.
